(19) United States
(12) Patent Application Publication
(10) Pub. No.: US 2002/0045260 A1
(43) Pub. Date: Apr. 18, 2002

(54) Method of isolating mesenchymal stem cells

(76) Inventors: Shih-Chieh Hung, Taipei (TW); Wai-Hee Lo, Taipei (TW)

Correspondence Address: Eric L. Prahl, Fish & Richardson P.C., 225 Franklin Street, Boston, MA, 02110-2804, US

(21) Appl. No.: 09/761,893

(22) Filed: Jan. 17, 2001

(30) Foreign Application Priority Data
Oct. 17, 2000 (TW) 89121676

(51) Int. Cl. C12N 5/08

(52) U.S. Cl. 435/368; 435/372

(57) ABSTRACT

The invention discloses a novel method of isolating mesenchymal stem cells (MSCs), which is characterized by purifying pluripotent MSCs based on physical characters and biological properties and without the uses of antibodies. The present invention also relates to the application of the isolated MSCs to serve as tissue replacement or gene therapy for tissues damaged by age, trauma, and disease.

### BACKGROUND OF THE INVENTION

1. Field of the Invention

The present invention relates to a novel and simple method of isolating, purifying, and culturally expanding mesenchymal stem cells, and to the characterizations and uses for such isolated mesenchymal stem cells.

2. Description of the Related Arts

With the advancement of cell culture and molecular biology, investigators have been rapidly developing new techniques and applications of tissue engineering (Langer R, Vacanti J P, Science 260:920-926, 1993) for disease treatment (Langer R, Vacanti J P, Scientific American 273(3):130-133, 1995). Three major components are involved in the application of tissue engineering in future clinical use, namely: (1) cells or stem cells; (2) biomaterial based scaffolds; and (3) bioactive factors (i.e. cytokines or growth factors). Stem cells are a population of cells found in embryos, fetus, and adult tissues that are capable of self-renewal in undifferentiated forms and regain the capability of multi-, pluri-, or toti-potential differentiation in conditioned environments.

Due to ethic problems, research and clinical studies using embryonic or fetus-derived stem cells are restricted or prohibited by laws in some countries. Furthermore, embryonic stem cells begin to differentiate into all kinds of tissues when implanted into nude mice, including dermis, muscle, bone, cartilage, and some unexpected components. For example, it is not ideal to have dermis, bone, and cartilage in induced tissues when only muscle is needed. On the other hand, adult-derived stem cells are easily controlled to differentiate into expected tissues and considered to have more promising applications in future uses.

Adult-derived stem cells comprise neural stem cells (NSCs), haematopoietic stem cells (HSCs), and mesenchymal stem cells (MSCs). MSCs are the primitive pluripotent blast cells found in bone marrow, blood, dermis, and periosteum. They can proliferate without differentiation and differentiate into any of the mesenchymal tissues (i.e. the tissues of the body that support the elements, comprising adipose, osseous, cartilaginous, elastic, and fibrous connective tissues) depending on various influences from cytokines or growth factors. Bone marrow, a complex tissue composed of a highly organized network of haematopoietic, endothelial, and mesenchymal cells, has also been used to treat bone or cartilage defects and degeneration (Vacanti Calif., et al., Transac. Orthop. Res. Soc. 18(1):276, 1993; Caplan A I, et al., Clin. Orthop. 342:254-269, 1997). MSCs from bone marrow that are culture-adherent have been hypothesized to contain osteoprogenitor cells, chondrogenitor cells (Weiss L, Anat. Rec. 186:161-184, 1976; Bianco P., Boyde A, Histochem. 100:93-99, 1993), and stem cells of adipogenic and fibroblastic cell lineages (Owen M E, Friedenstein A J, Stromal stem cells. In: Evered D., Harnett S (eds.) Cell and molecular biology vertebrate hard tissue, Ciba Found. Symp. Vol. 136. Willey, UK, pp.42-60, 1988; Caplan A I, Clin. Plastic Surg. 21:429-435, 1993).

Human MSCs having the capability of renewal and multilineage potential to differentiate into the adipogenic, chondrogenic, or osteogenic lineages have been isolated from marrow aspirates (Pittenger M F, et al., Science 2841:143-147, 1999). This is the first time that marrow-derived stem cells have been well manipulated ex vivo and denotes a new era of tissue engineering. Due to the lack of specific markers to immuno-select and the difficulties in developing a method to isolate, the application of bone marrow MSCs is limited. Bone marrow MSCs have been isolated via several methods. Friedenstein (Exp. Hematol. 4:276, 1976) placed whole bone marrow in culture plastic dishes and poured off the cells that were non-adherent after 4 hours. However, the isolated cells initially are heterogeneous and are difficult to clone. Several protocols are present now for isolation, including the use of STRO-1 (Oyajobi B O, et al., J. Bone Miner. Res. 14:351-361, 1999) and anti-Sca-1 (van Vlasselaer P, et al., Blood 84:753-763, 1994) monoclonal antibodies to isolate osteoprogenitor cells, and the indirect method by using anti-CD41 (Thiede M A, et al., U.S. Pat. No. 5,965,436) monoclonal antibodies to isolate megakaryocyte and megakaryocyte-associated MSCs.

All of these immuno-selection methods described above are complicated, mainly comprising the steps of: contacting cells with an antibody that specifically binds to the desired cells; separating the antibody-bound cells from unbound cells and removing unbound antibodies; and isolating or selecting desired cells that bind to the antibodies. Particularly, due to the adherence/interaction of the antibodies, the activity and surface antigens of the target cells may be affected or even destroyed (Basch R S, et al., J. Immunol. Methods 56:269, 1983). Further, the use of specific monoclonal or polyclonal antibodies will increase the time and cost for isolation. Thus, there is still a need for developing a method of isolating MSCs from bone marrow and other sources, which possesses the advantages of efficiency, simple operation, and low cost, so that the isolated MSCs can be largely used in the treatment of certain diseases.

### SUMMARY OF THE INVENTION

It is therefore the primary object of the present invention to provide a method for recovering mesenchymal stem cells by means of physical characteristics and biological properties. The method comprises: providing a mixture comprising mesenchymal stem cells; seeding the mixture into a culture device; and recovering and culturing the mesenchymal stem cells. Briefly, mesenchymal stem cells can be isolated with the use of a culture device depending on, for example, difference in cell size, different adherence capacity and the role of mesenchymal stem cells in supporting haematopoietic stem cells in co-culture.

The second object of the present invention is to provide an isolated mesenchymal stem cell recovered by the method as set forth above, which has the capability of self-renewal and pluripotent differentiation.

In one preferred embodiment, cell populations having greater than 98% of human MSCs can be obtained in accordance with the method of the invention, and such isolated MSCs can proliferate without differentiation and reach confluence even after 12 passages. The isolated MSCs of the present invention are uniform CD34⁻, and can be induced to differentiate into bone, adipose, cartilage, and various other type of connective tissues.

The third object of the present invention is to provide a composition comprising the isolated mesenchymal stem cells as mentioned above and a culture medium, wherein the medium expands the number of the mesenchymal stem cells.

The forth object of the present invention is to provide a pharmaceutical composition comprising the mesenchymal stem cells as mentioned above and a pharmaceutically acceptable carrier, wherein the mesenchymal stem cells are present in an amount sufficient to serve as tissue replacement or gene therapy for tissues damaged by age, trauma, and disease.

### BRIEF DESCRIPTION OF THE DRAWINGS

The present invention will be more fully understood and further advantages will become apparent when reference is made to the following description of the invention and the accompanying drawings in which:

FIG. 1(a) is a diagram showing cells have fibroblastic-like morphology after seeding of bone marrow cells into culture for 7 days; and FIG. 1(b) showing cells reach confluence with a consistent and homogenous morphology after seeding of bone marrow cells into culture for 17 days.

FIG. 2 is a diagram showing the flow cytometry analysis of cell homogeneity and specific response to CD surface markers.

FIG. 3 is a diagram showing osteogenic induction of human mesenchymal cell cultures, wherein the cells show varying degrees of positive stain for alkaline phosphatase in induction group 3(a) as compared to the controls 3(b).

FIG. 4 is a diagram showing the presence of mineral associated with the matrix after induction for 21 days, wherein the cells are stained by the von Kossa technique.

FIG. 5 is a diagram showing adipogenic differentiation of mesenchymal cell cultures, wherein the cells show varying degrees of positive stain for Oil-red O in induction group 5(a) as compared to the controls 5(b) after induction for 7 days.

FIG. 6 is a diagram showing chondrogenic differentiation of mesenchymal cell cultures, wherein cells show chondrocyte morphology in Safranin-O stain (a); and toludine blue stain (b) after induction for 21 days.

FIG. 7 is a diagram showing the presence of type II collagen in the matrix after induction for 21 days, wherein the cells are stained by immunohistochemistry.

### DETAILED DESCRIPTION OF THE INVENTION

Homogeneous populations of human mesenchymal stem cells (MSCs) are provided. MSCs serve as the progenitors for all mesenchymal cell lineages, including, but not limited to, osseous, adipose, cartilaginous, and fibrous connective tissues. Some surface markers of human MSCs from bone marrow may be verified by some monoclonal antibodies, such as SH2, SH3, and SH4 (see, for example, U.S. Pat. No. 5,486,359); nevertheless MSCs from bone marrow still lack specific markers with which to isolate or immuno-select them. Even using anti-CD41 monoclonal antibodies to isolate megakaryocyte and megakaryocyte-associated MSCs, the method is indirect and multiple steps are needed. Furthermore, the efficiency and purity of isolation remain to be improved.

Therefore, the present invention provides a novel, simple, effective, and economic method of isolating MSCs, comprising: providing a mixture comprising mesenchymal stem cells; seeding the mixture into a culture device; and recovering and culturing the mesenchymal stem cells.

The method of the present invention uses the physical characteristics and biological properties of MSCs to isolate MSCs from a cell mixture. More particularly, the method of the present invention relies on, for example, a difference in cell size, a difference in the capability of adhere and the role of MSCs in supporting the formation of haematopoietic colonies in co-culture with CD34⁺ haematopoietic stem cells. By means of their characteristics of large size (van Vlasselaer P, et al., supra), ease to adhere and their role in supporting haematopoietic stem cells (Huang S., et al., Nature 360:745, 1993), the method of the present invention was developed with the use of a culture device to physically isolate early MSCs.

In one preferred embodiment, the culture device comprises a plate with pores, wherein the pore size is sufficient for separating mesenchymal stem cells from other cells (e.g. haematopoietic stem cells). More preferably, the pore size ranges from about 0.4 to 40 microns in diameter.

According to the method of the present invention, any cells mixture containing MSCs can be the source materials for isolation. The source can derive from, for example, mammals (including human species), animals (e.g. rabbit), or plants. Suitable MSCs sources include, but are not limited to, fractioned tissues, un-fractioned tissues, bloods, or body fluids. Preferably, the MSCs sources include bone marrow, embryonic yolk sac, placenta, umbilical cord, and fetal, adolescent and adult body fluids and tissues, wherein the bone marrow can be obtained from iliac crest, femora, tibiae, spine, rib, or other medullary spaces.

The MSCs isolated by the method of the present invention possess the capability of self-renewal and pluripotent differentiation, which can be induced to differentiate into bone, adipose, cartilage, and various other type of connective tissues. In addition, the isolated MSCs of the present invention are uniform CD34⁻; however, MSCs with other types of surface markers are within the scope of the present invention.

According to the method of the present invention, medium that is useful in the culture and/or expansion of MSCs is not limited. One example of such medium used in the present invention is 10% fetal bovine serum (FBS) supplemented Dulbecco's modified Eagle's medium containing 1 g/L of glucose (DMEM-LG; Life Technologies). Other media and related additives, such as preservatives, pH indicators, are within the scope of the present invention.

After seeding bone marrow cells into the upper plate of the culture device which comprises pores with pore size ranging from about 0.4 to 40 microns in diameter therein, small-sized haematopoietic cells can pass through the pores in the plate to reach the plate base before adhering, and non-adherent cells can be removed by following changes of medium. Eventually, two kinds of cells can be recovered, respectively. The former, adhering to the lower dish with polygonal shape, lose the capability of renewal after subculture; the latter, adhering onto the upper plate with fibroblastic-like shape, have the capability of renewal and maintain the ability to differentiate into multiple lineages of mesenchymal tissues. The fibroblastic-like cells can be recovered with 0.25% trypsin-EDTA and re-seeded onto the culture dish in 10% FBS supplemented DMEM-LG without loss of the capacity to adhere. These isolated mesenchymal cell populations replicate without differentiation in 10% FBS supplemented DMEM-LG, and reach confluence at 17 days later following the first seeding.

In one aspect, the method of the present invention comprises the steps of providing a mixture containing MSCs; mingling the mixture with a ratio (e.g. 10%) of FBS supplemented medium; seeding the mixture and medium into a culture device; and recovering and culturing the MSCs in 10% FBS supplemented DMEM-LG. The isolated MSCs from the tissue specimen can maintain fibroblastic-like morphology and the capacity to adhere in the medium which contains factors that stimulate MSCs growth without differentiation and allow for the selective adherence of only the MSCs to substrate surface. The homogeneity of MSCs can then be attained by the removal of non-adherent cells from the dishes with the following changes of the medium.

In one preferred embodiment of the present invention, the isolated MSCs proliferate without differentiation and reach confluence even after 12 passages. The cell populations having greater than 98% homogeneous MSCs are obtained in accordance with the method of the present invention. Cells adhered onto the surface are fibroblastic-like in morphology and uniformly negative for CD34, CD14, CD38, CD50, CD120a in flow cytometry analysis. The desired cells in such cell populations are uniform CD34⁻ human MSCs that can be well distinguished from CD34⁺ haematopoietic stem cells and blood derived MSCs. In addition, the recovered MSCs can differentiate into bone, adipose, cartilage, and various other type of connective tissues under a suitable environment.

The present invention also comprises the application of such isolated MSCs, more particularly, in the form of pharmaceutical composition comprising a pharmaceutically acceptable carrier, which can be used for therapeutic or diagnostic purpose. For example, human MSCs are useful in: (1) providing an integral model of cell differentiation and tissue development to specific mesenchymal lineages; (2) developing mesenchymal cell lineages and assaying for factors associated with their differentiation and development; (3) detecting and evaluating growth factors or inhibitory factors which modulate MSCs proliferation and differentiation into specific mesenchymal lineages; (4) expanding a large scale of homogeneous/heterogeneous cells or tissues in vitro that can be implanted back into body combined with/without carriers, scaffolds, or bioactive factors such as cytokines; (5) producing various mesenchymal tissues for transplantation; (6) regenerating mesenchymal tissues which have been damaged by age, trauma, congenital, or acquired disease; and (7) genetically modulating culture-expanded MSCs ex vivo or in vitro to treat patients with mesenchymal tissue damages.

Without intending to limit it in any manner, the present invention will be further illustrated by the following examples.

### Example

### EXAMPLE 1. Bone Marrow Cell Preparation And Cell Culture

Bone marrow aspirates from the iliac crest were heparinized with 3,000 IU of heparin. The heparinized bone marrow was mixed with an equal volume of phosphate-buffered saline (PBS) and centrifuged at 900×g for 10 minutes at room temperature. The washed cells were resuspended in PBS to a final volume of 5 ml, and was layered over an equal volume of 1.073 g/ml Percoll solution, and then centrifuged at 900×g for 30 minutes. Mononuclear cells collecting at the interface were recovered. Percoll fractioned or unfractioned bone marrow cells in 10% fetal bovine serum (FBS) supplemented Dulbecco's modified Eagle's medium containing 1 g/L of glucose (DMEM-LG; Life Technologies) and antibiotics (penicillin 100 U/ml, and streptomycin 100 μg/ml) were seeded into the culture device at a density of 10⁶/cm². The cultures were maintained at 37° C. in 5% CO₂ in air, with medium changes first at 7 days after initial plating and then every 4 days.

### EXAMPLE 2. Isolation And Expansion of Mesenchymal Cells from Marrow

Symmetrical colonies of fibroblastic cells were visible at about 7 days after initial plating. Haematopoietic stem cells and non-adherent cells were removed with changes in medium. When the cultures reached confluence, cells were recovered with 0.25% trypsin-EDTA and re-plated at a density of 4×10³-10⁴/cm². The cells numbers were measured daily and the growth curve at different re-plating density were compared. The re-plating density with the best growing rate was used to expand the mesenchymal cells. The expanded cells were used for characterization of the renewal capacity and the specific response to a consistent set of surface marker antibodies.

### EXAMPLE 3. Flow Cytometry Analysis And Characterization

The isolated and expanded mesenchymal cells were characterized with monoclonal antibodies by flow cytometric analysis of specific surface antigens. The cells were harvested with the addition of 0.25% trypsin-EDTA, then washed twice with EDTA-PBS. The cells were exposed in 80 μl of 50×diluted FITC-conjugated human CD34 monoclonal antibody and then in 80 μl of 50×diluted one of PE-conjugated human CD monoclonal antibodies (CD14, CD29, CD38, CD50, and CD120a) with 1% BSA on ice for 45 minutes. The cell mixture was then washed twice with EDTA-PBS, and then fixed in 1% formaldehyde. Cells were analyzed with flow cytometry using a 525 nm bandpass filter for green FITC fluorescence and a 575 nm bandpass filter for red PE fluorescence.

### EXAMPLE 4. Induction of Multilineages Differentiation

The marrow mesenchymal cells 14 days following the first passage were cultured in DMEM-LG supplemented with 10% FBS. The cell culture was also treated with one of the following formulas: (1) osteogenic differentiation medium: 50 μg/ml of ascorbate-2-phosphate (Sigma Co.), 10⁻⁸ M of dexamethasone (Sigma Co.), and 10 mM of β-glycerophosphate (Sigma Co.); (2) adipogenic differentiation medium: 50 μg/ml of ascorbate-2-phosphate, 10⁻⁷ M of dexamethasone, and 50 μg/ml of indomethacin (Sigma Co.); (3) chondrogenic differentiation medium: 10 ng/ml TGF-β₁ in serum free aggregation condition. The medium was changed every 4 days and cells were used for histochemical or immunohistochemical analysis after the completion of differentiation by identified morphology.

### EXAMPLE 5. Histochemical Staining and Immunohistochemistry Study

The medium was removed from the culture and the cells were washed twice with PBS. The cells were fixed in 3.7% paraformaldehyde for 10 minutes at room temperature and washed with PBS. The cells treated by (1) formula were stained with alkaline phosphatase and von Kossa staining to reveal the osteogenic differentiation. Those treated by (2) and (3) formulas were stained with Oil red-O and Safranin-O or Toluidine blue to show the adipogenic differentiation and chondrogenic differentiation, respectively. Immunohistochemistry for human type II collagen was also made to demonstrate chondrogenic differentiation of bone marrow MSCs.

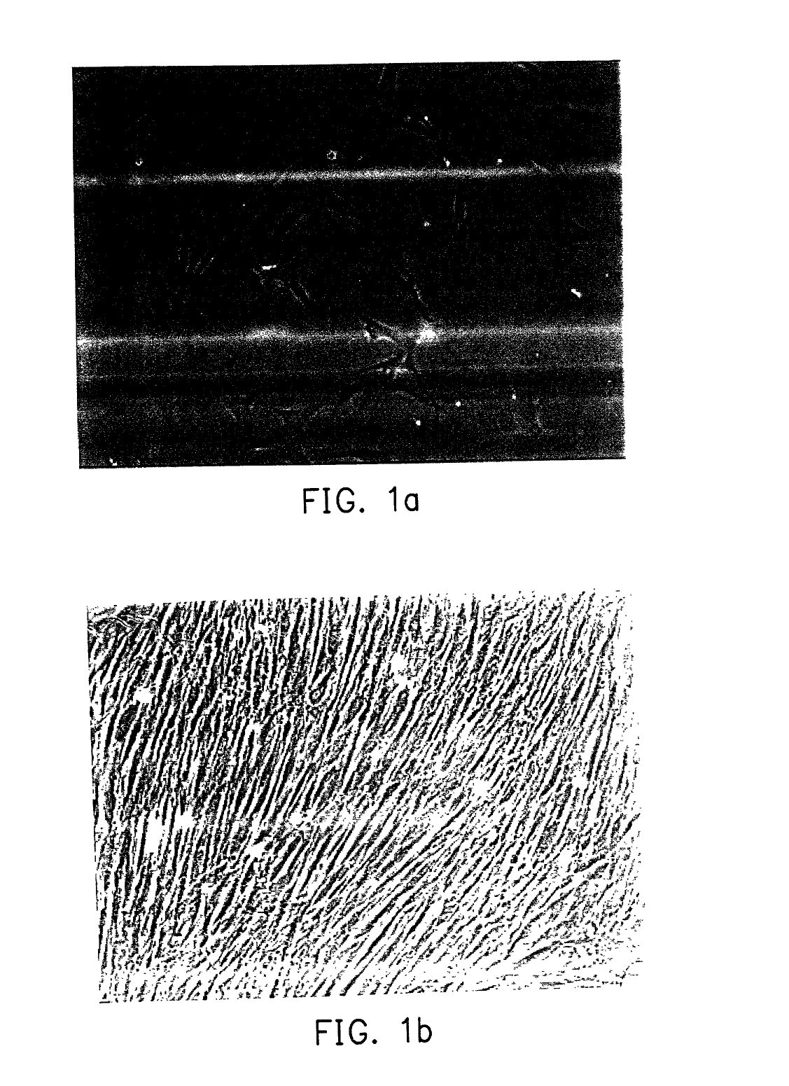

According to the method of the present invention, two kinds of adherent cells appeared when Percoll fractioned or un-fractioned bone marrow cells in 10% FBS supplemented DMEM-LG were seeded into the culture device. The early adherent cells present in the lower dishes are characterized by small size, polygonal shape, little renewal capacity, and were believed to be haematopoietic cells. The late adherent cells appearing several days later on the upper plate surface have fibroblastic-like morphology, the capacity to replicate, and the potential of multilineage differentiation, and were later confirmed to be MSCs. The late adhering cells maintained homogeneous morphology (referring to FIG. 1(a)) and were significantly greater in number than the same cells cultured by the conventional methods. In addition, the purification of these adherent cells was achieved by removal of the haematopoietic stem cells and non-adherent cells during the following changes of medium. The cells reach confluence 10 days later (referring to FIG. 1(b)), and cells re-plated at a ratio of 1:3 or at a density of 4×10³/cm² reach confluence 7 days later. The cells maintain a normal proliferation and undifferentiation status during culture expansion even at passage 12.

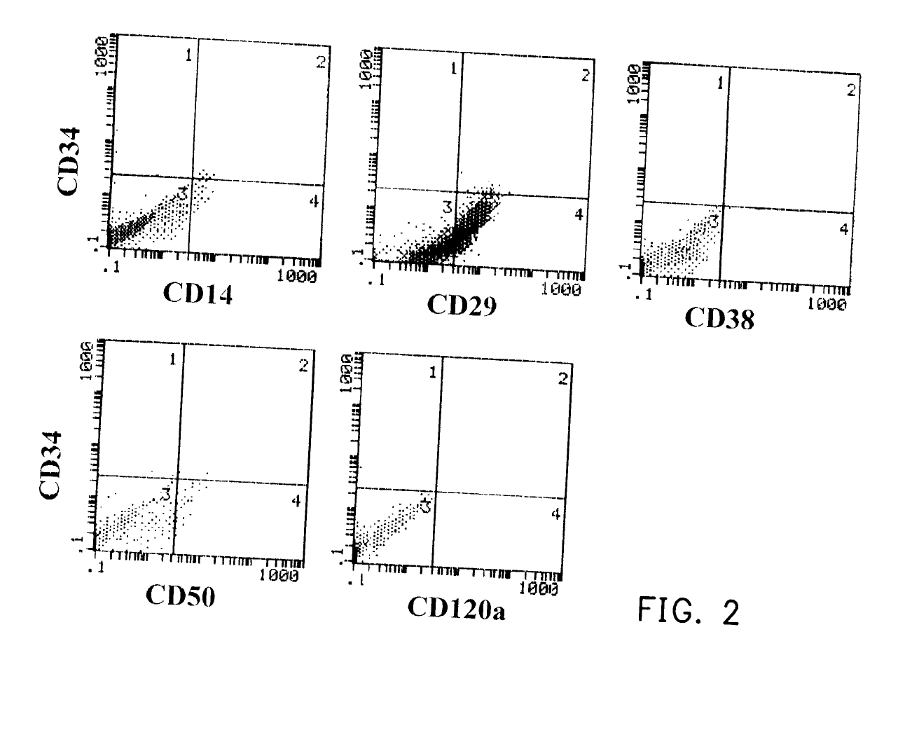

Bone marrow CD34⁺ fraction is considered to be haematopoietic stem cells, but CD34⁺, CD38⁻, HLADR⁻, CD50⁻, Stro-1⁺ fractions are reported to be stromal progenitors (Simmons P J, Torko-Storb B, Blood 78:2848, 1991; Waller E K, et al., Blood, 85:2422, 1995). Pittenger et al. (supra) suggests that bone marrow-derived MSCs are positive for CD44, CD71, CD90, CD106, CD120a, CD124, but negative for CD14, CD34, CD45. Referring to FIG. 2, the flow cytometry analysis indicates that these cells are a relatively homogeneous population at 14 days following the first passage. In addition, phenotypic characterization of the isolated MSCs reveals negative staining for CD14, CD34, CD38, CD50, and CD120a (which is positive in Pittenger's report), but low staining for CD29.

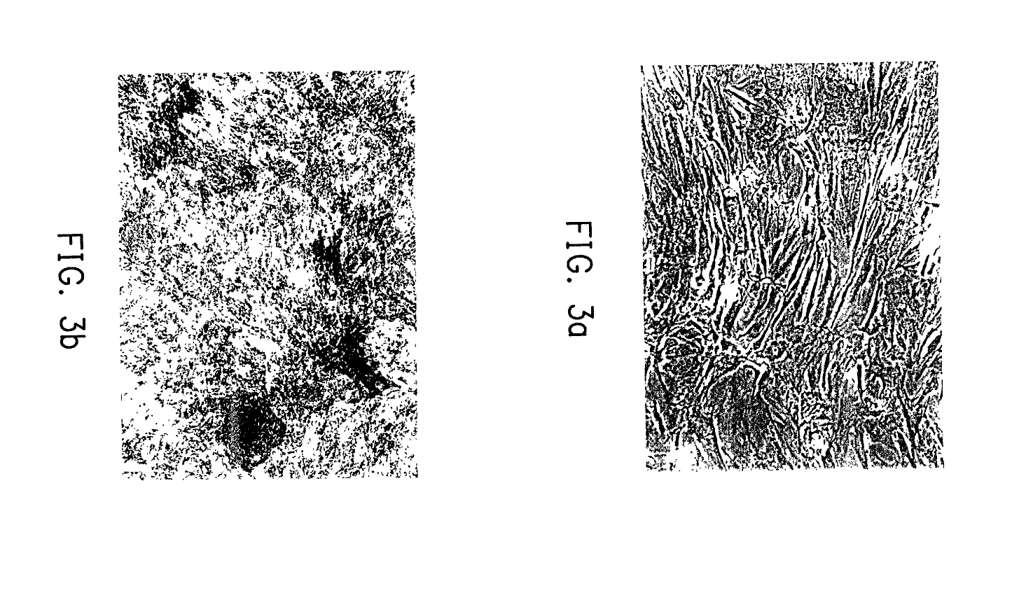

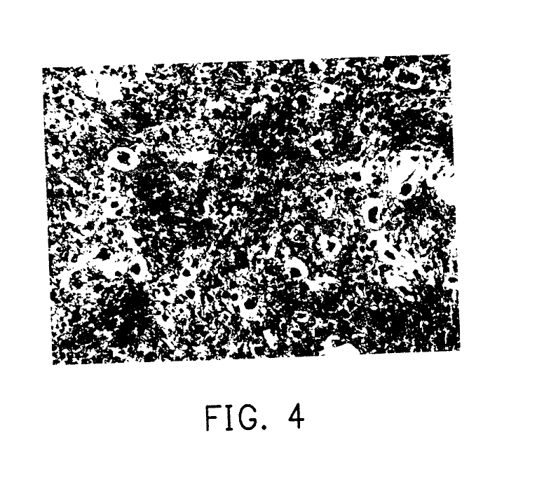

The osteogenic differentiation is attained at 16 days following the treatment. Under the influence of ascorbate, dexamethasone, and β-glycerophosphate, the isolated MSCs form alkaline phosphatase positive aggregates (FIG. 3) or von Kossa stain positive nodules (FIG. 4) as compared to the controls.

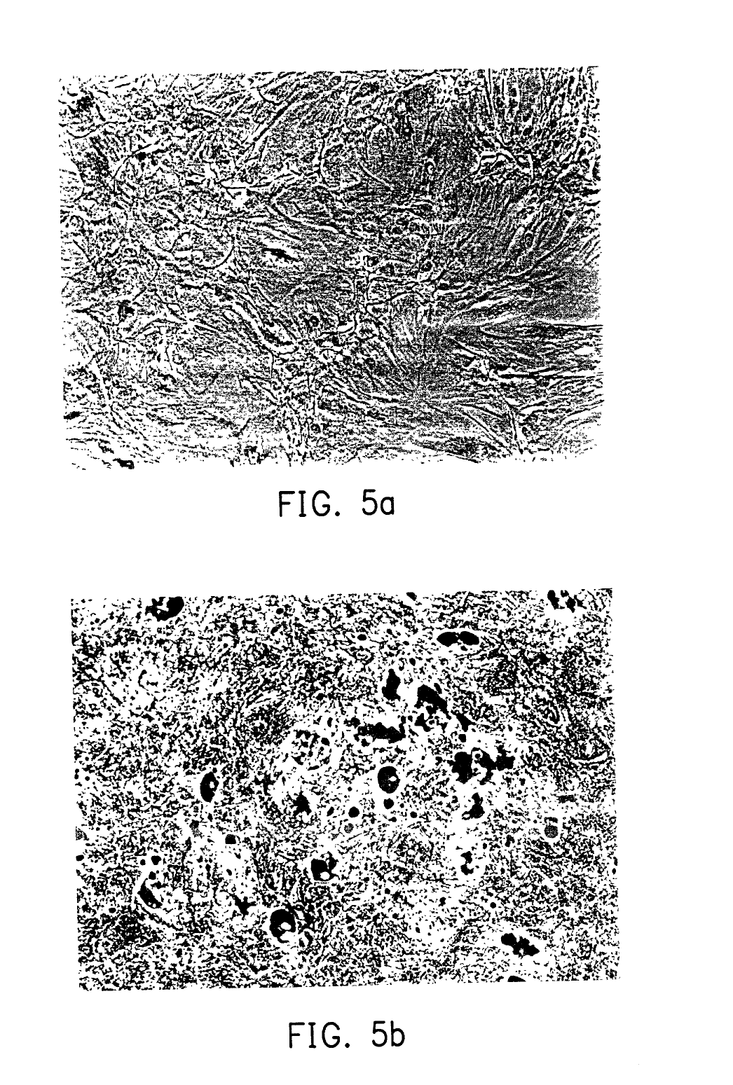

The adipogenic differentiation is achieved at 7 days following the treatment. Under the influence of ascorbate, dexamethasone, and indomethacin, the isolated MSCs form Oil red-O positive aggregates (FIG. 5) as compared to the controls. Adipogenic induction is also evident with the accumulation of lipid-rich vacuoles within cell with the eccentric deviation of the nucleus.

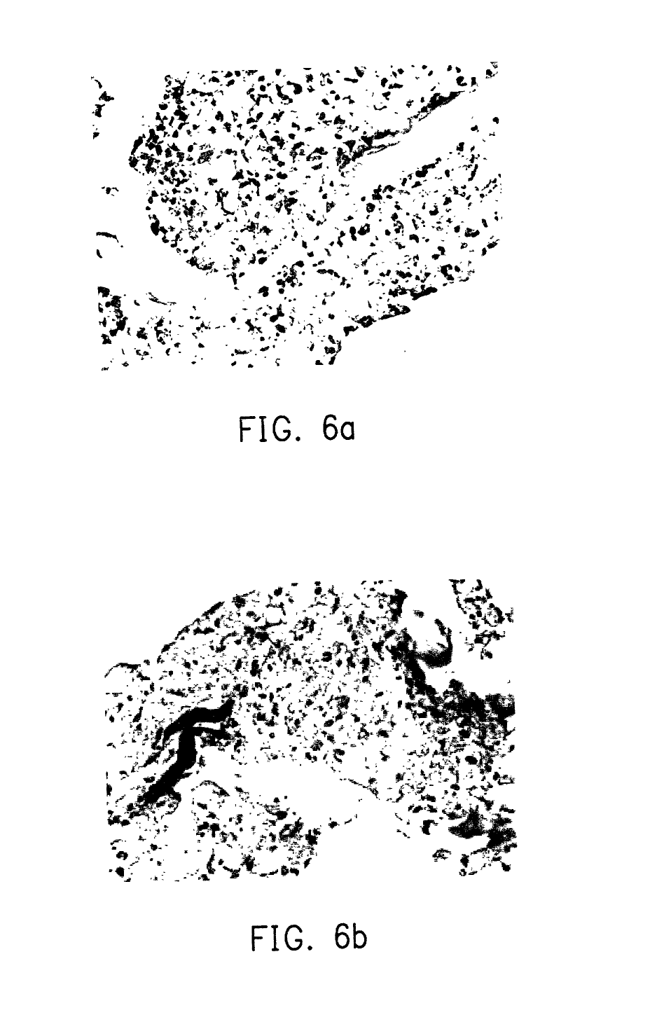

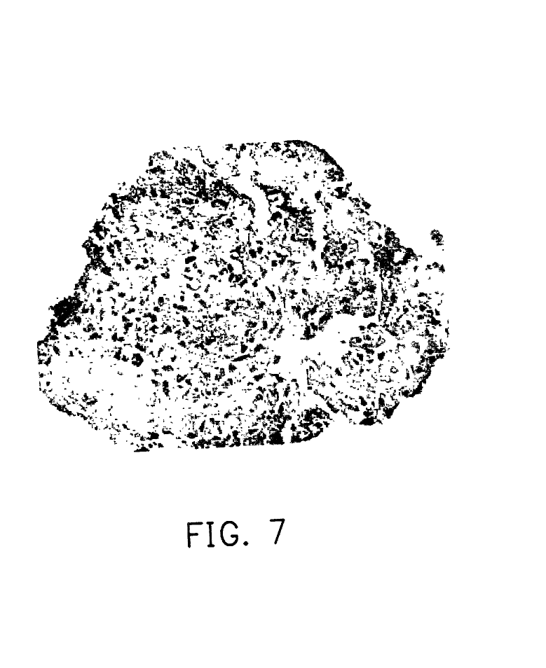

The chondrogenic differentiation is achieved at 21 days following the treatment. Positive Safranin O staining and Toludine blue staining with chondrocyte like lacunae and aggrecan- and type II collagen-rich extracellular matrix are evident in histological sections (FIG. 6) and immuno-histochemistry method (FIG. 7).

In conclusion, cell populations having greater than 98% of homogeneous human MSCs can be obtained in accordance with the method of the invention, and the isolated MSCs can proliferate without differentiation and reach confluence even after 12 passages. The isolated MSCs of the present invention are uniformed CD34⁻, and can be induced to differentiate into bone, adipose, cartilage, and various other type of connective tissues. Therefore, without the uses of any antibody to adsorb cells, the present invention provides an easy, simple, effective, and economic method to isolate and purify MSCs from bone marrow or other MSCs sources.

While the invention has been particularly shown and described with the reference to the preferred embodiment thereof, it will be understood by those skilled in the art that various changes in form and details may be made without departing from the spirit and scope of the invention.

What is claimed is:

1. A method for recovering mesenchymal stem cells, comprising:
(a) providing a mixture comprising mesenchymal stem cells;
(b) seeding the mixture into a culture device; and
(c) recovering and culturing the mesenchymal stem cells.

2. The method as claimed in claim 1, wherein said culture device comprises a plate with pores, wherein the pore size is sufficient for separating mesenchymal stem cells from other cells.

3. The method as claimed in claim 2, wherein the pore size ranges from about 0.4 to 40 microns in diameter.

4. The method as claimed in claim 1, wherein the mixture comprises cells selected from the group consisting of mammals, animals, and plants.

5. The method as claimed in claim 4, wherein the cells are selected from the group consisting of fractioned tissues, un-fractioned tissues, bloods, and body fluids.

6. The method as claimed in claim 5, wherein the mammal comprises human.

7. The method as claimed in claim 5, wherein the cells are selected from the group consisting of bone marrow, embryonic yolk sac, placenta, umbilical cord, and fetal, adolescent and adult body fluids and tissues.

8. The method as claimed in claim 1, wherein the mesenchymal stem cells have the capability of self-renewal and pluripotent differentiation.

9. The method as claimed in claim 8, wherein the mesenchymal stem cells can differentiate into tissues comprising bone, adipose, or cartilage.

10. The method as claimed in claim 8, wherein the mesenchymal stem cells are characterized by CD34⁻.

11. The method as claimed in claim 9, wherein the mesenchymal stem cells are cultured in DMEM-LG medium containing 10% fetal bovine serum.

12. An isolated mesenchymal stem cell recovered by the method as claimed in claim 1, which has the capability of self-renewal and pluripotent differentiation.

13. The mesenchymal stem cell as claimed in claim 12, which can differentiate into tissues comprising bone, adipose, or cartilage.

14. The mesenchymal stem cell as claimed in claim 12, which is characterized by CD34⁻.

15. A composition comprising the mesenchymal stem cell as claimed in claim 12 and a culture medium, wherein the medium expands the mesenchymal stem cell.

16. The composition as claimed in claim 15, wherein the mesenchymal stem cell is characterized by CD34⁻.

17. The composition as claimed in claim 15, wherein the medium comprises DMEM-LG medium containing 10% fetal bovine serum.

18. A pharmaceutical composition comprising the mesenchymal stem cell as claimed in claim 12 and a pharmaceutically acceptable carrier, wherein the mesenchymal stem cell is present in an amount sufficient to serve as tissue replacement or gene therapy for tissues damaged by age, trauma, and disease.

19. The pharmaceutical composition as claimed in claim 18, wherein the mesenchymal stem cell can differentiate into tissues comprising bone, adipose, or cartilage.

20. The composition as claimed in claim 18, wherein the mesenchymal stem cell is characterized by CD34⁻.
